(19) United States
(12) Patent Application Publication
(10) Pub. No.: US 2022/0409729 A1
(43) Pub. Date: Dec. 29, 2022

(54) COMPOSITION FOR ANTIMICROBIAL PHOTODYNAMIC THERAPY

(71) Applicant: CMS Dental A/S, Roslev (DK)

(72) Inventor: Jimmie Kert, Roslev (DK)

(21) Appl. No.: 17/776,486

(22) PCT Filed: Nov. 11, 2020
(86) PCT No.: PCT/EP2020/081797

§ 371 Date: May 12, 2022

(30) Foreign Application Priority Data
Nov. 13, 2019 (DK) PA201970694

(51) Int. Cl. A61K 41/00; A61K 8/20; A61K 8/22; A61K 8/49; A61K 8/67; A61N 5/06

(52) U.S. Cl. CPC A61K 41/0057; A61K 8/20; A61K 8/22; A61K 8/498; A61K 8/673; A61N 5/062

(57) ABSTRACT

The present invention relates to a composition and a kit of parts for antimicrobial photodynamic therapy and uses of the same, the composition comprising (a) hydrogen peroxide, carbamide peroxide and/or sodium hypochlorite and (b) at least one photosensitizer capable of being activated by light emitted in the blue spectrum, in particular curcumin, riboflavin and erythrosine.

### FIELD OF THE INVENTION

The present invention relates to a composition and a kit of parts for antimicrobial photodynamic therapy and uses of the same. The invention further relates a method of treating, ameliorating or reducing an infection or inflammation in the mouth cavity, such as gingivitis, periodontitis, peri-mucositis, peri-implantitis or endodontic infections.

### BACKGROUND OF THE INVENTION

Photodynamic therapy (PDT) is a form of phototherapy that involves light and a photosensitizing chemical substance, used in conjunction with molecular oxygen to elicit cell death, such as death of pathogenic microorganism.

Essentially, photodynamic therapy involves the use of non-toxic compound (porphyrins, chlorins or dyes) that act as photoactive drugs (referred to as photosensitizers) in combination with visible light of the appropriate wavelength to excite the photosensitizer. In the excited state, the photosensitizer, in the presence of the oxygen, transfers energy or electrons to ground state molecular oxygen producing reactive oxygen species (ROS) such as singlet oxygen and hydroxyl radical, which are responsible for the killing of cells.

Photodynamic therapy is an established modality for the treatment of cancer. It has also been extended for the treatment of noncancerous conditions such as age related macular degeneration and other dermatological applications. Besides these applications, there has also been a growing interest in the application of PDT for the treatment of infectious diseases. Elimination of pathogenic microorganisms by photodynamic therapy is termed antimicrobial PDT (aPDT). In the recent years, antimicrobial photodynamic therapy has drawn increasing attention from the dental practitioners for its potential in the treatment of oral infection, such periodontitis and endodontic infections.

There is a need in the art for improved compositions for use in dentistry and improved methods of antimicrobial photodynamic therapy.

### SUMMARY OF THE INVENTION

The object of the present invention is to provide improved compositions for use in dentistry such as improved composition for antimicrobial photodynamic therapy and methods for using such compositions.

A first aspect of the present invention provides a composition comprising
(a) hydrogen peroxide, carbamide peroxide, sodium hypochlorite or any combination thereof,
(b) at least one photosensitizer capable of being activated by light emitted in the blue spectrum, and
(c) optionally a gelling agent.

A second aspect of the present invention provides a kit of parts comprising
(a) a first container comprising a first composition comprising hydrogen peroxide, carbamide peroxide, sodium hypochlorite or any combination thereof,
(b) a second container comprising a second composition comprising at least one photosensitizer capable of being activated by light emitted in the blue spectrum, and
(c) optionally a source of light emitting light in the blue spectrum.

A third aspect of the present invention provides a composition or kits of parts of the present invention for use in the treatment of an infection in the mouth cavity, such as gingivitis, periodontitis, periimplantitis, perimucositis, periimplantitis or endodontic infections.

A further aspect of the present invention provides a method of treating, ameliorating or reducing an infection in the mouth cavity, said method comprising the steps of
(a) providing a composition comprising at least one photosensitizer according to the invention;
(b) applying the composition to the area of infection;
(c) activating photosensitizer(s) by illumination of the area of endodontic infection using a source of light emitting light in the blue spectrum.

### DETAILED DESCRIPTION OF THE INVENTION

In describing the embodiments of the invention specific terminology will be resorted to for the sake of clarity. However, the invention is not intended to be limited to the specific terms so selected, and it is understood that each specific term includes all technical equivalents which operate in a similar manner to accomplish a similar purpose.

In a first aspect, the present invention provides a composition comprising
(a) hydrogen peroxide, carbamide peroxide, sodium hypochlorite or any combination thereof,
(b) at least one photosensitizer capable of being activated by light emitted in the blue spectrum, and
(c) optionally a gelling agent.

One advantage of composition of the present invention is that treatment with the peroxide (hydrogen peroxide, carbamide peroxide, sodium hypochlorite or any combination thereof) and the photosensitizer(s) is performed simultaneously by co-administration. The treatment is thus faster and more comfortable for the patient.

The present inventor has discovered that the composition the peroxide (hydrogen peroxide, carbamide peroxide and/or sodium hypochlorite) and the photosensitizer(s) is stable for long-term storage.

The photosensitizer is a compound acceptable for application to the oral cavity. In the context of the present invention, the photosensitizer is a non-toxic compound (or at least non-toxic in the amount applied) that act as photoactive drugs in combination with visible light comprising light emitted in the blue spectrum to excite the photosensitizer. In one embodiment, the photosensitizer(s) is activated by the light in the range of 380 to 520 nm, such as 405 to 470 nm, preferably 455±30 nm. In another embodiment, the photosensitizer(s) has a peak absorption around 455 nm, preferably 455±30 nm.

The photosensitizer is typically a porphyrin, chlorin or dye. In one embodiment, the photosensitizer(s) is a natural (non-synthetic) photosensitizer. In another embodiment, the photosensitizer(s) is selected from the group consisting of curcumin, riboflavin and erythrosine. In a further embodiment, the photosensitizer is curcumin. In yet an embodiment, the photosensitizer is riboflavin.

In one embodiment, the composition comprises least two photosensitizers, such as two, three, four or five photosensitizers capable of being activated by light emitted in the blue spectrum. In one embodiment, the composition comprises curcumin and riboflavin. In another embodiment, the composition comprises two photosensitizers, which are curcumin and riboflavin.

The composition comprises photosensitizer(s) in amounts sufficient to provide a therapeutic effect (treating, ameliorating or reducing the infection) in the dose applied to the infected area, for example a tooth pocket or a root canal of an infected tooth. In one embodiment, the composition comprises 0.001 to 0.1 wt % of said at least one photosensitizer, such as 0.01 to 0.1 wt % of the at least one photosensitizer. In one embodiment, the composition comprises 0.1 wt % of the at least one photosensitizer, such as 0.1 wt % curcumin, 0.1 wt % riboflavin, or a total of 0.1 wt % curcumin and riboflavin. In another embodiment, the composition comprises 0.01 wt % of the at least one photosensitizer, such as 0.01 wt % curcumin, 0.01 wt % riboflavin, or a total of 0.02 wt % curcumin and riboflavin. In further embodiment, the composition comprises 0.03 wt % of the at least one photosensitizer, such as 0.02 wt % curcumin, 0.01 wt % riboflavin, or a total of 0.03 wt % curcumin and riboflavin.

The composition of the present invention comprises hydrogen peroxide, carbamide peroxide, sodium hypochlorite or any combination thereof. In a preferred embodiment, the composition comprises hydrogen peroxide.

Hydrogen peroxide, carbamide peroxide and sodium hypochlorite work by producing destructive hydroxyl free radicals that can attack membrane lipids, DNA, and other essential cell components. Catalase, produced by aerobic organisms and facultative anaerobes that possess cytochrome systems, can protect cells from metabolically produced the peroxides by degrading the peroxides to water and oxygen. This defence mechanism is overwhelmed by the concentrations used for disinfection.

Hydrogen peroxide, carbamide peroxide and sodium hypochlorite have demonstrated antimicrobial activity against a wide range viruses, bacteria, yeasts, and bacterial spores. In general, greater activity is seen against Gram-positive than Gram-negative bacteria. Under normal conditions, Hydrogen peroxide, carbamide peroxide and sodium hypochlorite are stable when properly stored (e.g., in dark containers). The decomposition or loss of potency in small containers is low at ambient temperatures.

In one embodiment, the composition comprises 0.25 to 5 wt % of hydrogen peroxide, carbamide peroxide or sodium hypochlorite or any combination thereof, such as 0.25 to 5 wt % of hydrogen peroxide, carbamide peroxide or sodium hypochlorite or any combination thereof, such as 0.25 to 5 wt % of hydrogen peroxide or 0.25 to 5 wt % of sodium hypochlorite. In one embodiment, the composition comprises hydrogen peroxide in the range of 2.5 to 4 wt % hydrogen peroxide, for example 2.5 to 3.5 wt % hydrogen peroxide, preferably 3 wt % hydrogen peroxide or about 3 wt % hydrogen peroxide.

The hydrogen peroxide may be provided in the form of carbamid peroxide. Carbamide peroxide consist equal molar amounts of hydrogen peroxide (molar weight of 34.01 g/mol) and urea (molar weight of 60.06 g/mol). Thus, where the composition comprises 0.25 to 5 wt % of hydrogen peroxide and the hydrogen peroxide is provided in the form of carbamid peroxide, the composition further comprises about 0.44 to 8.8 wt % of urea. In one embodiment, the composition comprises carbamid peroxide corresponding to 2.5 to 4 wt % hydrogen peroxide (and thus about about 4.4 to 7.1 wt % of urea), for example 2.5 to 3.5 wt % hydrogen peroxide (and thus about about 4.4 to 6.2 wt % of urea), preferably 3 wt % hydrogen peroxide or about 3 wt % hydrogen peroxide (and thus about about 5.3 wt % of urea).

Accordingly, in one embodiment, the composition comprises 0.69 to 13.8 wt % of carbamid peroxide. In one embodiment, the composition comprises carbamid peroxide in the range of 6.9 to 11.1 wt % carbamid peroxide, for example 6.9 to 9.7 wt % carbamid peroxide, preferably 8.3 wt % carbamid peroxide or about 8.3 wt % carbamid peroxide.

In another embodiment, the composition comprises 3 wt % or about 3 wt % hydrogen peroxide. In another embodiment, the composition comprises 2.5 wt % or about 2.5 wt % sodium hypochlorite.

In order to treat periodontal disease, it may not be optimal or sufficient to deliver the composition into the infected periodontal pocket(s). The order to hold the disposed composition in the infected periodontal pocket(s) for sufficient time to activate the composition using the source of light (emitting light in the blue spectrum) and allowing the composition to work (e.g. debride the cell walls of pathogenic bacteria) it may be advantageous to apply the composition as an aqueous gel. Thus in one embodiment, composition comprises a gelling agent. In another embodiment, the composition comprises a gelling agent selected from the group consisting of guar gum, gum acacia, tragacanth, sodium alginate, pectin, starch, gelatin, cellulose derivative (such as hydroxypropyl methyl cellulose, methylcellulose and sodium carboxymethyl cellulose), carbomers, polyvinyl pyrrolidone, polyvinyl alcohol and xanthan gum or any combination thereof. In one embodiment, the gelling agent is guar gum.

The amount of gelling agent in the composition may dependent on the gelling agent and the preferred viscosity of the gel. The composition typically comprises 0.5% to 10 wt % of said gelling agent, for example about 7% to 10 wt % of of said gelling agent, for example about 7% to 9 wt % of of said gelling agent or such as 1, 2, 3, 4, 5, 6, 7, 8, or 9 wt % of said gelling agent. The composition is typically provided in dry form for storage, such as a powder, which may be reconstituted to an aqueous gel briefly before use.

In a further embodiment, the composition comprises hydrogen peroxide, riboflavin, curcumin and guar gum. For example, about 3 wt % hydrogen peroxide, about 0.1 wt % curcumin, about 0.1 wt % riboflavin and about 0.5% to 10 wt % of guar gum, such as about 7% to 10 wt % of guar gum, for example about 7% to 9 wt % of guar gum, such as about 8 wt % of guar gum. In another embodiment, the composition comprises about 3 wt % hydrogen peroxide, about 0.01 wt % curcumin, about 0.01 wt % riboflavin and about 0.5% to 10 wt % of guar gum, such as about 7% to 10 wt % of guar gum, for example about 7% to 9 wt % of guar gum, such as about 8 wt % of guar gum. In a further embodiment, the composition comprises about 3 wt % hydrogen peroxide, about 0.02 wt % curcumin, about 0.01 wt % riboflavin and about 0.5% to 10 wt % of guar gum, such as about 7% to 10 wt % of guar gum, for example about 7% to 9 wt % of guar gum, such as about 8 wt % of guar gum. In one embodiment, the composition is provided in dry form, which may be reconstituted to an aqueous gel before use.

The composition may further comprises one or more additional acceptable excipients such as one or more of a preservative, one or more antioxidants, and/or a surfactant, which may contribute for example to the stability of the composition.

A second aspect of the present invention provides a kit of parts
(a) a first container comprising a first composition comprising hydrogen peroxide, carbamide peroxide, sodium hypoclorite or any combination thereof,
(b) a second container comprising a second composition comprising at least one photosensitizer capable of being activated by light emitted in the blue spectrum, and
(c) optionally a source of light emitting light in the blue spectrum.

One advantage of using photosensitizer(s) capable of being activated by light emitted in the blue spectrum is that dental curing light equipment that is used for polymerization of light cure resin based composites typically emits light in the blue spectrum. Thus, often no further instruments need to be integrated or around in the work station of the dentist in order to perform the antimicrobial photodynamic therapy. Thus, in a preferred embodiment, the kits of part is provided without a source of light, in particular where a suitable source of light is already available in the dental clinic.

In another embodiment, the kit comprises at least one photosensitizers selected from the group consisting of curcumin, riboflavin and erythrosine. In another embodiment, composition comprises two photosensitizers, such as curcumin and riboflavin.

In one embodiment, the first composition of said kit comprises hydrogen peroxide, such as 0.25 to 5 wt % hydrogen peroxide, such as 2.5 to 4 wt % hydrogen peroxide, for example 2.5 to 3.5 wt % hydrogen peroxide, preferably 3 wt % hydrogen peroxide or about 3 wt % hydrogen peroxide.

In another embodiment, the first and/or said second composition comprises a gelling agent. In a further embodiment, the gelling agent is selected from the group consisting of guar gum, gum acacia, tragacanth, sodium alginate, pectin, starch, gelatin, cellulose derivative (such as hydroxypropyl methyl cellulose, methylcellulose and sodium carboxymethyl cellulose), carbomers, polyvinyl pyrrolidone, polyvinyl alcohol and xanthan gum. In one embodiment, the first and/or said second composition comprises 0.5% to 10 wt % of said gelling agent, for example about 7% to 10 wt % of of said gelling agent, for example about 7% to 9 wt % of of said gelling agent or such as 1, 2, 3, 4, 5, 6, 7, 8, or 9 wt % of said gelling agent.

In one embodiment, the first and second composition of the kit is in dry form, such as a powder.

In a third aspect, the invention provides the composition or kits of parts of the invention for use in the treatment of an infection in the mouth cavity, such as a dental infection.

In one embodiment, the composition or kits of parts is for use in the treatment of gingivitis, periodontitis, periimplantitis, endodontic infection. In one embodiment, the composition or kits of parts is for use in endodontic photoactivated disinfection.

A further aspect of the present invention provides a method of treating, ameliorating or reducing an infection in the mouth cavity, said method comprising the steps of
(a) providing a composition of the present invention comprising at least one photosensitizer;
(b) applying the composition to the area of infection;
(c) activating photosensitizer(s) by illumination of the area of infection using a source of light emitting light in the blue spectrum.

The infection subject to treatment or amelioration may for example be gingivitis, periodontitis, periimplantitis, endodontic infection. In one embodiment, the infection is in a periodontal pocket or a dental root canal.

The order to hold the disposed composition in the infected periodontal pocket or dental root for sufficient time composition may be applied in the form of an aqueous gel. Thus, in one embodiment, the composition is a gel and the gel is applied to one or more periodontal pockets.

The source of light may be any source of light emitting sufficient light in the blue spectrum to activate the photosensitizer(s). In one embodiment, the the area of infection is illuminated with said light with an illumination intensity in the range of around 2000 to 7000 mW/cm², such as in the range of 2000 to 6000 mW/cm², for example in the range of 2000 to 5000 mW/cm², such as in the range of 2000 to 4000 mW/cm², for example 2000 to 3000 mW/cm², such as 3000 mW/cm². In one embodiment, the area of infection is illuminated with said light with an illumination intensity of around 2000 mW/cm² or around 12 J/cm². In another embodiment, the area of infection is illuminated with said light for at least 30 seconds, such as 60 seconds, for example 1 minute, such as about 2 minutes, for example about 3 minutes, such as about 4 minutes, for examples about 5 minutes. In a third embodiment, the area of infection is illuminated with said light with an illumination intensity of around 2000 mW/cm² or around 12 J/cm² and the area of infection is illuminated with said light for at least 30 seconds, such as 60 seconds, for example 1 minute, such as about 2 minutes, for example about 3 minutes, such as about 4 minutes, for examples about 5 minutes.

In another embodiment, the light emitted from said source of light comprises light in the range of 380 to 520 nm, preferably 455±30 nm.

As mentioned, the source of light may be any source of light emitting sufficient light in the blue spectrum to activate the photosensitizer(s). In one embodiment, light is a blue light emitting diode (LED) or a blue light emitting optical fibre. In another embodiment, a dental curing light device that is also used for polymerization of light cure resin based composites typically emits light in the blue spectrum.

The infection in the mouth cavity typically comprises one or more one or more periodontal pathogenic microorganism. The infection microorganism may be present as biofilm and or a planktonic infection. In one embodiment, the infection is primarily in the form of biofilm, such as a biofilm comprising one or more periodontal pathogenic microorganism.

In one embodiment, pathogenic microorganism is selected from the group consisting of bacteria, fungi and virus. In another embodiment, the periodontal pathogenic microorganism is a bacterium. In one embodiment, the microorganism is a gram-negative bacterium. In one embodiment, the microorganism is a gram-positive bacterium.

In one embodiment, the one or more microorganisms is selected from the group consisting of Porphyromonas gingivalis, Tannerella forsythia, Fusobacterium nucleatum, Enterococcus faecalis, Streptococcus intermedius, Streptococcus mutans, Streptococcus gordonii, Prevotella intermedia and Actinomyces naeslundii. In another embodiment, the microorganisms is or comprises Porphyromonas gingivalis.

When describing the embodiments of the present invention, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.

The terms “comprising”, “comprise” and “comprises” herein are intended by the inventors to be optionally substitutable with the terms “consisting of”, “consist of” and “consists of”, respectively, in every instance.

1. A composition comprising
(a) hydrogen peroxide, carbamide peroxide and/or sodium hypochlorite,
(b) at least one photosensitizer selected from the group consisting of curcumin and riboflavin, and
(c) optionally a gelling agent.

2-4. (canceled)

5. The composition according to claims 1, wherein said composition comprises at least two photosensitizers.

6. The composition according to claim 2, wherein said composition comprises curcumin and riboflavin.

7. (canceled)

8. The composition according to claim 1, wherein said composition comprises 0.25 to 5 wt % of hydrogen peroxide, carbamide peroxide, sodium hypoclorite, or any combination thereof.

9. The composition according to claim 1, wherein said composition comprises 0.25 to 5 wt % hydrogen peroxide.

10. (canceled)

11. The composition according to claim 1, wherein said composition comprises 0.5% to 10 wt % of said gelling agent.

12. The composition according to claim 1, wherein said composition comprises hydrogen peroxide, riboflavin, curcumin and guar gum.

13. The composition according to claim 7, wherein said composition comprises about 3 wt % hydrogen peroxide, about 0.02 wt % curcumin, about 0.01 wt % riboflavin and about 0.5% to 10 wt % of guar gum

14-15. (canceled)

16. The composition according to claim 1, wherein said composition further comprises a preservative, one or more antioxidants, and/or a surfactant.

17. A kit of parts
(a) a first container comprising a first composition comprising hydrogen peroxide, carbamide peroxide, sodium hypoclorite or any combination thereof,
(b) a second container comprising a second composition comprising at least one photosensitizer capable of being activated by light emitted in the blue spectrum, wherein the at least one photosensitizer is selected from the group consisting of curcumin and riboflavin, and
(c) optionally a source of light emitting light in the blue spectrum.

18. (canceled)

19. The kit according to claim 17, wherein said composition comprises curcumin and riboflavin.

20. The kit according to claim 17, wherein said first composition comprises hydrogen peroxide, such as 0.25 to 5 wt % hydrogen peroxide.

21. The kit according to claim 17, wherein said first and/or said second composition comprises a gelling agent.

22. (canceled)

23. The kit according to according to claim 21, wherein said composition comprises 0.5% to 10 wt % of said gelling agent.

24-28. (canceled)

29. A method of treating, ameliorating or reducing an infection in the mouth cavity, said method comprising the steps of
(a) providing a composition comprising at least one photosensitizers according claim 1;
(b) applying the composition to the area of infection;
(c) activating at least one photosensitizer by illumination of the area of endodontic infection using a source of light emitting light in the blue spectrum.

30. The method of claim 29, wherein said infection is in a periodontal pocket or a dental root canal.

31. (canceled)

32. The method according to claim 30, wherein said composition is a gel and wherein said gel is applied to one or more periodontal pockets.

33. The method according to claim 29, wherein the light emitted from said source of light comprises light in the range of 380 to 520 nm.

34-35. (canceled)

36. The method according to claim 29, wherein said area of said endodontic infection is illuminated with said light with an illumination intensity of around 2000 to 7000 mW/cm2.

37. (canceled)

38. The method according to claim 29, wherein said infection comprises one or more periodontal pathogenic microorganism selected from the group consisting of Porphyromonas gingivalis, Tannerella forsythia, Fusobacterium nucleatum, Enterococcus faecalis, Streptococcus intermedius, Streptococcus mutans, Streptococcus gordonii, Prevotella intermedia and Actinomyces naeslundii.

39-44. (canceled)
